(19) United States
(12) Patent Application Publication
(10) Pub. No.: US 2023/0255508 A1
(43) Pub. Date: Aug. 17, 2023

(54) ARTIFICIAL INTELLIGENCE (AI) AIDED COVID - 19 DIAGNOSTIC KIT

(71) Applicant: NEW SENSES UZAY TEKNOLOJI VE SAGLIK ARASTIRMALARIA, Istanbul (TR)

(72) Inventor: Oguzhan AYDEMIR, Istanbul (TR)

(21) Appl. No.: 18/015,734

(22) PCT Filed: Nov. 4, 2020
(86) PCT No.: PCT/TR2020/051040

§ 371 Date: Jan. 12, 2023

(30) Foreign Application Priority Data
Jul. 12, 2020 (TR) 2020/11037

(51) Int. Cl. A61B 5/08; A61B 5/00; A61B 5/097

(52) U.S. Cl. CPC A61B 5/082; A61B 5/0022; A61B 5/002; A61B 5/097; A61B 5/742; A61B 5/7264; A61B 2560/0214

(57) ABSTRACT

The invention relates to a smart device (A) developed to detect HCO3, pO2, pCO2, and EtOH on alveolar odor and thus the Covid-19 positiveness, instead of the arterial blood gas values detected in blood samples taken over the radial, brachial and femoral arteries, without the need for blood or biological swabs.

### TECHNICAL FIELD

The present invention relates to a device developed to detect HCO3, pO2, pCO2, and EtOH on alveolar odor and thus the Covid-19 positiveness, instead of the arterial blood gas values detected in blood samples taken over the radial, brachial and femoral arteries, without the need for blood or biological swabs.

### PRIOR ART

The human lung is exposed to a large number of airborne pathogens as a result of inhaling 10,000 liters of air per day. The observation that respiratory infections are still rare is evidence of the presence of an effective host defense system on the mucosal surface of the lung. The issue regarding that these host defense system and pathogens cause some odors by producing ethanol, etc., is discussed in the medical literature and is the subject of various studies. For example, diabetes mellitus leads to the accumulation of ketone bodies and their excretion by respiration. This odor is a typical one and resembles the odor of acetone. In addition, in cases that lead to diabetes and other insulin resistance, there is a decrease in body secretions, especially saliva, that this dryness of the mouth also contributes to halitosis. In tumoral lesions observed in any part of the body, bad breath occurs as a result of the volatile gases formed due to the necrotic process being expelled with tidal air. Especially in ovarian cancers and in the early diagnosis of some other malignancies, malignant melanoma, lung cancer, breast cancer, there are studies in the literature conducted using trained dogs and electronic noses. Although some studies have shown that electronic noses are used to distinguish the said odors, cacosmia meters, in which decision support systems equipped with artificial intelligence have been tried, have not been developed yet.

Although there is an application regarding the Covid-19, called “Covid-19 infection healing method” and numbered 2020/05195 in the Turkish Patent database, the patent is related to healing and not related to diagnostics.

In another application numbered 2019/16368 and called “Kit for detection of large six Enterohemorrhagic Escherichia coli serotypes by RT-PCR”, the invention relates to PCR diagnostic kits and is about the diagnosis of diseases by swabbing or drawing blood.

Another application regarding the technique is the application numbered 2019/01360. The invention named “Electronic Nose System for Early Diagnosis and Detection of Attacks of Hereditary Metabolic Patients” is about the low-cost electronic nasal system that enables the early diagnosis of hereditary metabolic diseases and the detection of attacks by measuring the odor by transferring the air drawn from the aero chamber containing a mask and spray chamber to the sample container.

### THE PURPOSE OF INVENTION

The purpose of the invention is to develop a device to detect HCO3, pO2, pCO2, and EtOH on alveolar odor and thus the Covid-19 positiveness, instead of the arterial blood gas values detected in blood samples taken over the radial, brachial and femoral arteries, without the need for blood or biological swabs.

Another purpose of the invention is to use artificial intelligence supported biosensors as a new test method that is easy to use in general clinical and Covid-19 diagnosis. Thus, these devices will be produced and thus they facilitate the diagnosis of Covid-19 and accelerate the treatment process.

Another purpose of the invention is that the test kit, called CovidNois, is designed to instantly perform a special scoring for covid-19 by analyzing data identified by artificial intelligence. In this way, the control physician will be able to diagnose the disease in a shorter time.

Another aim of the invention is to introduce a method that will allow arterial blood gas impressions of Covid-positive patients to be made without taking blood.

The invention is a mobile device that will support diagnosis and treatment for Covid-19 and can provide information about metabolic alkalosis, and metabolic acidosis, and Arterial Blood Gas rating with artificial intelligence.

Arterial blood gas (ABG) is one of the most fundamental laboratory methods that provide reliable information about the metabolic and respiratory status of patients. Indications for ABG sampling can be listed as follows;

A. Assessment of the diagnosis, follow-up, and response to treatment of acid-base balance disorder in a patient,

B. Assessment of the diagnosis, follow-up, and response to treatment of respiratory failure in a patient,

C. To be able to explain the reason of sudden and/or unexplained pathologies such as shortness of breath and confusion.

In order to fulfill the purposes described above, the invention is a set of systems that analyze the alveolar air with the reactions resulting from electrical signals in the reaction room inside the device and calculate reference values with the formula given with the artificial intelligence software embedded in the hardware.

### BRIEF DESCRIPTION OF THE FIGURES

FIG. 1 is an overall view of the device of the invention.

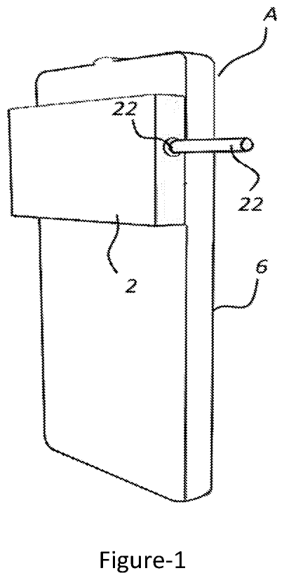

FIG. 2 is a view showing the distribution of the components inside the body of the device of the invention.

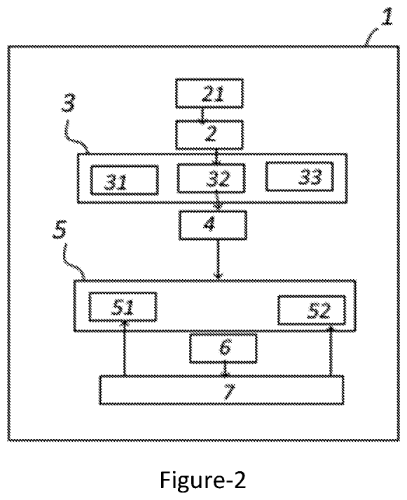

FIG. 3, is the flow chart of the device of the invention.

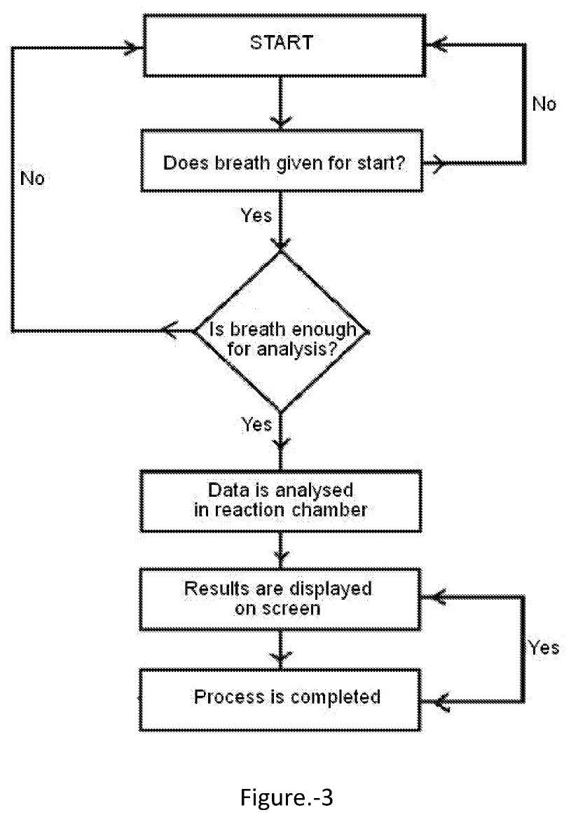

### REFERENCE NUMBERS

A—Device
1—Central Control Unit
2—Pipette Hopper
21—Pipette Inlet
22—Pipette
3—Reaction Chamber
31—Moisture Sensor
32—Oxygen Sensor
33—Hydrogen Sensor
4—Programmable digital processor
5—Adapter Chamber
51—Wifi Module
52—Bluetooth Module
6—Display
7—Battery

### DETAILED DESCRIPTION OF THE INVENTION

The device (A) of the invention consists of; the central control unit (1) that modulates the data, the pipettes inlet (21) that allows the alveolar air to be blown in, the pipettes reservoir (2) where the breath is concentrated before being taken to the reaction chamber (3), the moisture sensor (31), the oxygen sensor (32), and the hydrogen sensor (33), respectively, in the reaction chamber (3), a programmable digital processor (4) that interprets the reaction chamber data and converts it into numerical data, the display showing the data (6) and the Bluetooth module (52) sharing the data with the desired device and located in the adapter chamber (5) and the wireless module (51) that shares the data with the cloud server and the battery (7) that provides energy to the circuit.

Therefore, together with its at least one pipette inlet (21) that allows the alveolar air to be blown in, at least one pipette inlet (21) where the pipette (22) is positioned, at least one pipette chamber (2) where the breath is concentrated before being taken to the reaction chamber (3), with at least one humidity sensor (31), at least one oxygen sensor (32), and at least one hydrogen sensor (33) where alveolar air contacts alternately in the reaction chamber (3), the device has the ability to detect HCO3, pO2 and pCO2 from arterial blood gases.

The working principle of the device (A); Human blood contains about 0.003 promille alcohol (which means that a person weighing 80 kg has 1.68 grams of pure alcohol). But this value increases in covid-19 positive patients.

Therefore, when the formula pK (6.1)+logHCO3−(24)/EtOH) is applied, a result in the range of 215-457 values indicates covid-19 positivity. The calculation also requires that the person participating in the test has not consumed alcohol for 48 hours.

The device (A) is a system that captures HCO3, pO2 and pCO2 molecules from alveolar odor by means of the intermediary components, determines the reference range of the captured gas molecules, and enables the diagnosis of Covid-19. And also, by its system with constantly learning software, it is capable of calculating arterial blood gas values and blood pH values and diagnosing Covid-19.

1. The invention is a device (A) that can diagnose covid-19 via alveolar odor, without the need for blood or biological swabs, instead of arterial blood gas values detected in blood samples taken over radial, brachial and femoral arteries, wherein; comprising,
a central control unit (1) that modulates data,
a pipette (22) that allows alveolar air to enter the device by blowing,
at least one reaction chamber (3) and
a programmable digital processor (4) that makes sense of data and converts it into numeric data.

2. The invention is a device (A) according to claim 1, wherein; said reaction chamber (3) comprises the moisture sensor (31), the oxygen sensor (32), and the hydrogen sensor (33) in all which the alveolar air contact.

3. The invention is a device (A) according to claim 1, wherein; comprising a pipette chamber (2) in which the breath is concentrated before being taken into the said reaction chamber (3).

4. The invention is a device (A) according to claim 1, wherein; comprising at least one display (6) showing the data generated through the said digital processor (4).

5. The invention is a device (A) according to claim 1, wherein; comprising an adapter chamber (5) and a Bluetooth module (52) that shares data with the desired device.

6. The invention is a device (A) according to claim 1, wherein; comprising a wireless module (51) that shares data with the cloud server.

7. The invention is a device (A) according to claim 1, wherein; comprising a battery (7) that provides energy to the circuit.

8. The invention is a device (A) according to claim 1, wherein; comprising at least one pipette inlet (21) where the said pipette (22) is placed.
